(19) United States
(12) Patent Application Publication
(10) Pub. No.: US 2019/0021914 A1
(43) Pub. Date: Jan. 24, 2019

(54) CONTROLLING SURFACE DISPERSIBILITY IN THERMOPLASTIC INJECTION MOLDED AND FLUSHABLE MATERIALS

(71) Applicant: Kimberly-Clark Worldwide, Inc., Neenah, WI (US)

(72) Inventors: Alphonse DeMarco, Seal Harbour (CN); Gregory J. Wideman, Menasha, WI (US); Mark M. Mleziva, Appleton, WI (US)

(21) Appl. No.: 15/756,144

(22) PCT Filed: Oct. 30, 2017
(86) PCT No.: PCT/US17/58998

§ 371 Date: Feb. 28, 2018

Related U.S. Application Data
(60) Provisional application No. 62/414,948, filed on Oct. 31, 2016.

(51) Int. Cl. A61F 13/26

(52) U.S. Cl. CPC A61F 13/266; C08J 7/123

(57) ABSTRACT

An injection-molded article includes a water-dispersible injection-moldable composition including 82 wt. % to 86 wt. % partially-hydrolyzed polyvinyl alcohol (PVOH), 11 wt. % to 13 wt. % plasticizer, and 3 wt. % to 5 wt. % total colorant and slip additives, wherein the injection-molded article has an outer surface, and wherein the composition at the outer surface is surface cross-linked. A method for controlling the dispersibility of an injection-molded article having an outer surface includes formulating a water-dispersible injection-moldable composition including 82 wt. % to 86 wt. % partially-hydrolyzed polyvinyl alcohol (PVOH), 11 wt. % to 13 wt. % plasticizer, and 3 wt. % to 5 wt. % total colorant and slip additives; injection molding the single resin composition into the injection-molded article; and treating the outer surface to increase the cross-linking of the composition at the outer surface.

### BACKGROUND

The present disclosure relates generally to tampon applicators. Vaginal tampons are disposable absorbent articles sized and shaped (e.g., cylindrical) for insertion into a women's vaginal canal for absorption of body fluids generally discharged during the woman's menstrual period. Insertion of the tampon into the vaginal canal is commonly achieved using a tampon applicator that comes initially assembled with the tampon.

Tampon applicators are typically of a two-piece construction, including a barrel in which the tampon is initially housed and a plunger moveable telescopically relative to the barrel to push the tampon out of the barrel and into the vaginal canal. The barrel has a tip that generally retains the tampon within the barrel until pushed through the tip by the plunger. In normal use, the applicator and more particularly the barrel of the applicator is held by the user by gripping one portion of the barrel (e.g., toward the trailing or plunger end of the barrel) and inserting the barrel, tip end first, into the vaginal canal. The barrel is pushed partially into the canal so that a portion (e.g., toward the leading or exit end of the tampon barrel) is disposed within the vaginal canal and is contact with the walls lining the canal. The plunger is then used to push the tampon out through the tip of the barrel and into the canal. The plunger and barrel are then removed from the vaginal canal, leaving the tampon in place.

Flushable feminine care products provide consumers with discretion and convenience benefits. Current plastic tampon applicators, however, are made of injection molded materials such as polyolefins (e.g., polypropylenes or polyethylenes) and polyesters that are not biodegradable or renewable, as the use of biodegradable polymers in an injection molded part is problematic due to their high cost and to the difficulty involved with thermally processing such polymers. As a result, consumers must dispose of tampon applicators in a separate waste receptacle, which results in a challenge for consumers to dispose of the applicators in a discrete and convenient manner. Furthermore, the soiled or used tampon applicator can also pose a biohazard or potential health hazard. Although current plastic tampon applicators are not supposed to be flushed, some consumers can nevertheless attempt to flush the applicators in the toilet, which can lead to clogging of sewer pipes and municipal waste water treatment facilities. Attempts have been made to mold cold water-dispersible materials such as poly(vinyl alcohol) (PVOH) to alleviate these problems, but such attempts have not been successful. Instead, when using PVOH in tampon applicators, the materials must be solution processed so that they can be formed into a tampon applicator that has a thick enough wall, and such solution processing is a slow, costly, environmentally-unsustainable process that necessitates high energy requirements. Further, although cardboard applicators have been developed, the cardboard must often be coated to decrease the coefficient of friction of the applicator to a comfortable level for consumers, and the coatings used are not environmentally friendly and add to the costs associated with forming the applicator.

The challenge in producing a flushable tampon applicator is that materials that disperse in water also tend to degrade when exposed to humidity/water in air and to the moisture inherent in mucosal linings.

As such, a need currently exists for a thermoplastic, water-dispersible composition that can be injection molded, where such compositions can be successfully formed into a tampon applicator. A need also exists for a water-dispersible applicator that is comfortable to insert and that does not begin to break down upon insertion or during storage.

### SUMMARY

In one aspect, an injection-molded article includes a water-dispersible injection-moldable composition including 82 wt. % to 86 wt. % partially-hydrolyzed polyvinyl alcohol (PVOH), 11 wt. % to 13 wt. % plasticizer, and 3 wt. % to 5 wt. % total colorant and slip additives, wherein the injection-molded article has an outer surface, and wherein the composition at the outer surface is surface cross-linked.

In an alternate aspect, a method for controlling the dispersibility of an injection-molded article having an outer surface includes formulating a water-dispersible injection-moldable composition including 82 wt. % to 86 wt. % partially-hydrolyzed polyvinyl alcohol (PVOH), 11 wt. % to 13 wt. % plasticizer, and 3 wt. % to 5 wt. % total colorant and slip additives; injection molding the single resin composition into the injection-molded article; and treating the outer surface to increase the cross-linking of the composition at the outer surface.

Objects and advantages of the disclosure are set forth below in the following description, or can be learned through practice of the disclosure.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a perspective view of one aspect of a water-dispersible tampon applicator as contemplated by the present disclosure;

FIG. 2 is a schematic view of a representative injection molding apparatus used to manufacture the tampon applicator of FIG. 1;

FIG. 3 is a schematic plan view of a standard test sample mold used in the present application;

FIG. 4 is a graphical illustration demonstrating the Effect of Dose on Physical Properties;

FIG. 5 is a graphical illustration demonstrating the Physical Properties of Irradiated PVOH Tampon Tubes;

FIG. 6 is a graphical illustration demonstrating the Contact Angle of Exposed and Non-exposed Surfaces;

FIG. 7 is a graphical illustration demonstrating the Physical Properties of Irradiated PVOH with MBA Tampon Tubes;

FIG. 8 is a graphical illustration demonstrating the Contact Angle Comparison of PVOH with MBA after Exposure;

FIG. 9 is a graphical illustration demonstrating the Slosh Box Dispersibility of PVOH plus MBA;

FIG. 10 is a graphical illustration demonstrating the Coefficient of Friction of Exposed and Non-exposed Disc Sides; and

FIG. 11 is a graphical illustration demonstrating the Dispersibility of Discs Irradiated on Two Sides.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

Generally speaking, the present disclosure is directed to a thermoplastic composition that is water-sensitive (e.g., water-soluble, water-dispersible, etc.) in that it loses its integrity over time in the presence of water, yet also has a high enough melt flow index and a low enough melt viscosity such that it can be molded into an article such as a tampon applicator. For instance, the thermoplastic composition has a high enough melt flow index and a low enough melt viscosity such that it can be injected molded. The composition contains partially-hydrolyzed PVOH and a plasticizer. The desired water-sensitive attributes and mechanical properties of the composition and the resulting molded articles, such as tampon applicators, can be achieved in the present disclosure by selectively controlling a variety of aspects of the composition, including the nature of each of the components employed, the relative amount of each component, the ratio of the weight percentage of one component to the weight percentage of another component, and the manner in which the composition is formed. A tampon applicator is described herein as a specific example of an article that can be produced under the present disclosure, but developments described herein are equally applicable to any type of molded article.

### I. APPLICATOR DESIGN

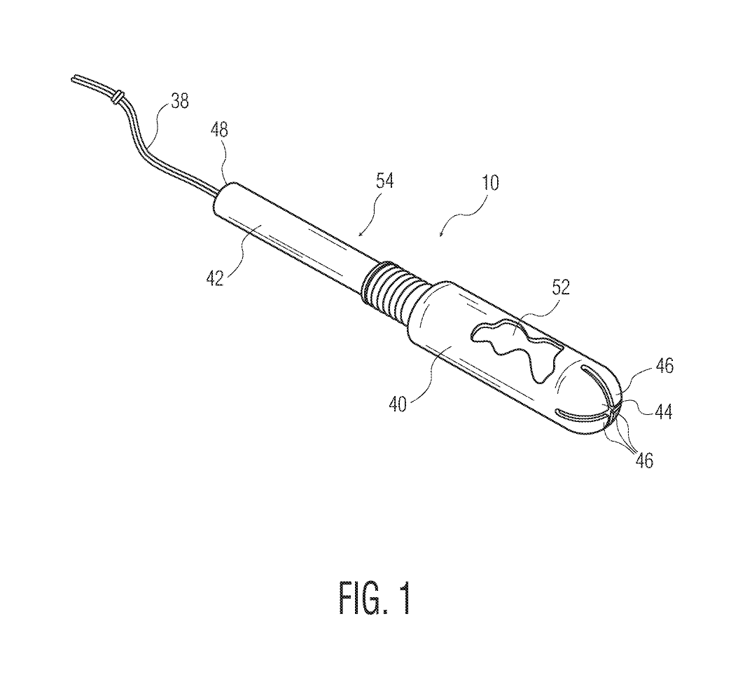

As illustrated in the tampon assembly 10 of FIG. 1, the tampon applicator 54 comprises an outer tube 40 and an inner tube 42. The outer tube 40 is sized and shaped to house a tampon 52. A portion of the outer tube 40 is partially broken away in FIG. 1 to illustrate the tampon 52. In the illustrated embodiment, the outer tube 40 has a substantially smooth exterior surface, which facilitates insertion of the tampon applicator 54 without subjecting the internal tissues to abrasion. The outer tube 40 may be coated to give it a high slip characteristic. The illustrated outer tube 40 is a straight, elongated cylindrical tube. It is understood however that the applicator 54 could have different shapes and sizes than those illustrated and described herein.

Extending outwardly from the outer tube is an insertion tip 44. The insertion tip 44, which is formed as one-piece with the outer tube 40, may be dome-shaped to facilitate insertion of the outer tube into a woman's vagina in a comfortable manner. The illustrated insertion tip 44 is made of a thin, flexible material and has a plurality of soft, flexible petals 46 that are arranged to form the dome-shape. The petals 46 are capable of radially flexing (i.e., bending outward) to provide an enlarged opening through which the tampon 52 can exit when it is pushed forward by the inner tube 42. It is to be understood, however, that the outer tube 40 may be formed without the insertion tip 44. Without the insertion tip, the outer tube includes an opened end (not shown) through which the tampon 52 can exit when it is pushed forward by the inner tube.

The inner tube 42 is an elongate cylinder that is used to engage the tampon 52 contained in the outer tube 40. A free end 48 of the inner tube 42 is configured so that the user can move the inner tube with respect to the outer tube 40. In other words, the free end 48 functions as a grip for the forefinger of the user. The inner tube 42 is used to push the tampon 52 out of the outer tube 40 and into the woman's vagina by telescopically moving into the outer tube. As the inner tube 42 is pushed into the outer tube 40 by the user, the tampon 52 is forced forward against the insertion tip 44. The contact by the tampon 52 causes the petals 46 of the insertion tip 44 to radially open to a diameter sufficient to allow the tampon to exit the outer tube 40 and into the woman's vagina. With the tampon 52 properly positioned in the woman's vagina, the tampon applicator 54 is withdrawn. In a used configuration of the tampon applicator 54, the inner tube 42 is received in the outer tube 40.

The inner tube 42, the outer tube 40, and the insertion tip 44 can be formed from one or more layers, where one layer includes the water-dispersible, thermoplastic composition of the present invention. Further, to prevent the applicator 54 from prematurely disintegrating due to moisture during use and/or to reduce the coefficient of friction of the applicator 54 to make it more comfortable for the user, it can be coated with a water-insoluble material that also has a low coefficient of friction to enhance comfort and prevent disintegration during insertion of the applicator 54. The structure of the tampon applicator described above is conventional and known to those skilled in the art, and is described, for instance, in U.S. Pat. No. 8,317,765 to Loyd, et al., which is incorporated herein in its entirety by reference thereto for all purposes. Other tampon applicator structures that can be formed from the thermoplastic composition of the present invention are described, for instance, in U.S. Pat. No. 4,921,474 to Suzuki, et al. and U.S. Pat. No. 5,389,068 to Keck, as well as U.S. Patent Application Publication Nos. 2010/0016780 to VanDenBogart, et al. and 2012/0204410 to Matalish, et al., which are incorporated herein in their entirety by reference thereto for all purposes.

Generally speaking, frictional forces occur between any two contacting bodies where there are forces tending to slide one of the bodies relative to the other. The frictional forces act parallel to the contacting surfaces and opposite the forces tending to cause sliding between the bodies. Further, the frictional forces are proportional to normal forces on the bodies and to the tendency of the bodies to grip each other.

As used herein, the coefficient of friction is the ratio of the frictional force between the bodies to the normal force between the bodies. The coefficient of friction is different between bodies at rest and bodies moving relative to each other. In general, two bodies contacting one another, but not moving relative to one another, will exhibit greater frictional resistance to motion than bodies that are moving relative to one another. Hence, a static coefficient of friction (i.e., a coefficient of friction between bodies that are not moving relative to each other) can but need not necessarily be somewhat greater than a dynamic coefficient of friction (i.e., a coefficient of friction between bodies that are moving relative to each other). Larger coefficients of friction correspond to larger amounts of friction between bodies, while smaller frictional coefficients correspond to smaller amounts of friction. As used further herein, the term coefficient of friction refers to at least one of a static coefficient friction and a dynamic coefficient of friction. In particularly suitable aspects, the coefficient of friction differential described previously is present for both static and dynamic coefficients of friction.

One or more additives can be added to the polymeric first layer 81 of the barrel 23 (prior to molding) to enhance the slip characteristic (e.g., to provide a low coefficient of friction) of the barrel outer surface at least at the central region 43 of the barrel and more suitably at the central region and tip region 45 of the barrel. For example, suitable such additives include without limitation erucamide, dimethicone, oleamide, fatty acid amide and combinations thereof. It is understood that other additives can used to provide enhanced slip characteristics to the barrel 23 outer surface without departing from the scope of this disclosure. In other aspects the barrel 23 can instead, or additionally, be coated with a friction reducing, or slip agent such as, without limitation, wax, polyethylene, silicone, cellophane, clay and combinations thereof. In still other suitable aspects the barrel 23 can include a polymer blend melted together and co-extruded to provide a low coefficient of friction.

In the illustrated aspect, the barrel 23 is further constructed so that the barrel outer surface at the tip region 45 has a lower coefficient of friction than at the central region 43 of the barrel to facilitate easier insertion of the barrel, inner end first, into the vaginal canal. This is particularly useful on days that a period is relatively light. For example, the outer surface of the barrel 23 at the tip region 45 can be configured to have a substantially lower surface roughness than at the central region 43 of the barrel, and more suitably the tip region can be substantially smooth or polished to reduce the coefficient of friction of the tip region relative to that of the central region. As a particular example, the surface roughness (that provides a tactile perception to the user) of the central region 43 of the barrel can have a surface roughness of less than or equal to about 36 and is more suitably about 27 in accordance with VDI Richtlinie [Standard] 3400. VDI Richtlinie 3400 has the German title: “Electroerosive Bearbeitung, Begriffe, Verfahren, Anwendung” [Electrical Discharge Machining, Definitions, Process, Application], published by the Verein Deutscher lngenieure [Association of German Engineers] in June 1975.

In other aspects, the tip region 45 of the barrel 23 can instead, or additionally be coated with a friction reducing agent so that the outer surface of the barrel at the tip region has a lower coefficient of friction than that of the central region of the barrel. Providing a surface roughness differential between the tip region 45 and the central region 43 also serves as a visual indicator of the reduced friction coefficient at the tip region.

### II. APPLICATOR MATERIALS

As described above, a water-dispersible injection-moldable resin for use in a flushable tampon applicator of the structure described herein is needed. All previous attempts to make a flushable injection molded tampon applicator have failed because the material could not be injection molded at low cycle times, and because the applicators were difficult to insert under moist conditions or had poor shelf-lives under high-moisture conditions. This disclosure allows for the successful production of a flushable tampon applicator that provides consumers a clean experience by eliminating the messiness of applicator disposal.

PVOH is a water-soluble, repulpable, and biodegradable resin with excellent aroma and oxygen barrier properties and resistance to most organic solvents. The polymer is used extensively in adhesives, textile sizing, and paper coating. Despite its excellent mechanical, physical, and chemical properties, the end uses of PVOH have been limited to those uses in which it is supplied as a solution in water. This limitation is partly due to the fact that vinyl alcohol polymers in an unplasticized state have a high degree of crystallinity and show little to no thermoplasticity before the occurrence of decomposition that starts at about 170° C. and becomes pronounced at 200° C., which is well below its crystalline melting point.

Attempts have been made to use PVOH in injecting molding for disposable sanitary products such as tampon applicators. These can yield molded parts that are stiff when removed from the molding machine but pick up moisture from the atmosphere and become too flexible for machine handling in the manufacture of tampon applicators. Other attempts use complex mixtures of materials, multiple types of PVOH, and/or various coatings. Tampon applicators made primarily from PVOH are water-dispersible and biodegradable; however, such applicators have been shown to suffer from issues involving moisture sensitivity, stability, odor, and stickiness. Hence there have been no commercially successful launches of these applicators.

Other attempts in addressing the flushability of plastic tampon applicators include plastic applicators made from other water-soluble materials such as polyethylene oxide polymers, thermoplastic starch, and hydroxypropyl cellulose; plastic tampon applicators made from combinations of water-soluble and water-insoluble/biodegradable materials such as combinations of PVOH and polycaprolactone, combinations of polyethylene oxide and polycaprolactone, combinations of polyethylene oxide and polyolefins such as polypropylene and polyethylene; and combinations of PVOH and polyethylene oxide polymers. Again, none of these attempts to produce a truly flushable product have seen commercial application.

A water-dispersible injection-moldable resin based on PVOH has been developed for use as the primary resin for injection molding outer and inner (plunger) tubes in current tampon applicators. The resin is a blend of single low molecular weight partially-hydrolyzed PVOH and a plasticizer such as glycerin. In addition, the applicator resin formulation can include other materials such as color additives, antioxidants, surface finish, and release agents/lubricants such as a euricamide release agent.

A single grade of PVOH, specifically a PVOH partially hydrolyzed at 87-89%, with a low molecular weight provides the speed of dispersibility required for flushability. This PVOH is plasticized with glycerin to adjust the melt flow rate to be compatible with injection molding. The level of plasticizer is low enough that it does not bloom during storage, which would result in an unusable product. The plasticizer level also contributes to the softness or hardness of the final product.

A. Polyvinyl Alcohol Polymer

The water-dispersible, thermoplastic composition includes one or more polymers containing a repeating unit having a functional hydroxyl group, such as polyvinyl alcohol (“PVOH”) and copolymers of PVOH (e.g., ethylene vinyl alcohol copolymers, methyl methacrylate vinyl alcohol copolymers, etc.). Vinyl alcohol polymers, for instance, have at least two or more vinyl alcohol units in the molecule and can be a homopolymer of vinyl alcohol or a copolymer containing other monomer units. Vinyl alcohol homopolymers can be obtained by hydrolysis of a vinyl ester polymer, such as vinyl formate, vinyl acetate, or vinyl propionate. Vinyl alcohol copolymers can be obtained by hydrolysis of a copolymer of a vinyl ester with an olefin having 2 to 30 carbon atoms, such as ethylene, propylene, or 1-butene; an unsaturated carboxylic acid having 3 to 30 carbon atoms, such as acrylic acid, methacrylic acid, crotonic acid, maleic acid, or fumaric acid or an ester, salt, anhydride or amide thereof; an unsaturated nitrile having 3 to 30 carbon atoms, such as acrylonitrile or methacrylonitrile; a vinyl ether having 3 to 30 carbon atoms, such as methyl vinyl ether or ethyl vinyl ether; and so forth. The degree of hydrolysis can be selected to optimize solubility, for example, of the polymer. For example, the degree of hydrolysis can be from about 60 mole % to about 95 mole %, in some aspects from about 80 mole % to about 90 mole %, in some aspects from about 85 mole % to about 89 mole %, and in some aspects from about 87 mole % to about 89 mole %. These partially-hydrolyzed PVOHs are cold-water soluble. In contrast, completely-hydrolyzed or nearly-hydrolyzed PVOHs are not soluble in cold water.

Examples of suitable partially-hydrolyzed PVOH polymers are available under the designations SELVOL 203, 205, 502, 504, 508, 513, 518, 523, 530, or 540 PVOH from Sekisui Specialty Chemicals America, LLC of Dallas, Tex. For instance, SELVOL 203 PVOH has a percent hydrolysis of 87% to 89% and a viscosity of 3.5 to 4.5 centipoise (cps) as determined from a 4% solids aqueous solution at 20° C. SELVOL 205 PVOH has a percent hydrolysis of 87% to 89% and a viscosity of 5.2 to 6.2 cps as determined using a 4% solids aqueous solution at 20° C. SELVOL 502 PVOH has a percent hydrolysis of 87% to 89% and a viscosity of 3.0 to 3.7 cps as determined using a 4% solids aqueous solution at 20° C. SELVOL 504 PVOH has a percent hydrolysis of 87% to 89% and a viscosity of 4.0 to 5.0 cps as determined from a 4% solids aqueous solution at 20° C. SELVOL 508 PVOH has a percent hydrolysis of 87% to 89% and a viscosity of 7.0 to 10.0 cps as determined as determined from a 4% solids aqueous solution at 20° C. Other suitable partially-hydrolyzed PVOH polymers are available under the designations ELVANOL 50-14, 50-26, 50-42, 51-03, 51-04, 51-05, 51-08, and 52-22 PVOH from DuPont. For instance, ELVANOL 51-05 PVOH has a percent hydrolysis of 87% to 89% and a viscosity of 5.0 to 6.0 cps as determined from a 4% solids aqueous solution at 20° C.

In the present disclosure, the PVOHs characterized as having a low viscosity include SELVOL 502 PVOH (3.0 to 3.7 cps), where the midpoint or average viscosity for low-viscosity PVOH is generally less than about 3.35 cps, as determined by averaging the minimum and maximum viscosities provided for commercially available partially-hydrolyzed PVOHs. The PVOHs characterized as having a high viscosity include SELVOL 203 PVOH (3.5 to 4.5 cps), SELVOL 504 PVOH (4.0-5.0 cps), ELVANOL 51-05 PVOH (5.0 to 6.0 cps), SELVOL 205 PVOH (5.2 to 6.2 cps), and SELVOL 508 PVOH (7.0-10.0 cps), where the midpoint or average viscosity for the high-viscosity PVOH polymers is at least about 4.0 cps, as determined by averaging the minimum and maximum viscosities provided for commercially-available partially-hydrolyzed PVOHs.

B. Plasticizer

A plasticizer is also employed in the water-dispersible thermoplastic composition to help render the water-soluble polymer thermoplastic and thus suitable for extrusion into pellets and subsequent injection molding. Suitable plasticizers include, for instance, polyhydric alcohol plasticizers such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, and sorbitol), polyols (e.g., ethylene glycol, glycerol, propylene glycol, dipropylene glycol, butylene glycol, and hexane triol), and polyethylene glycols. Also suitable are hydrogen-bond-forming organic compounds that do not have a hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable plasticizers can include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids can also be used, such as ethylene acrylic acid, ethylene maleic acid, butadiene acrylic acid, butadiene maleic acid, propylene acrylic acid, propylene maleic acid, and other hydrocarbon-based acids. A low molecular weight plasticizer is preferred, such as less than about 20,000 g/mol, preferably less than about 5,000 g/mol, and more preferably less than about 1,000 g/mol.

The plasticizer can be incorporated into the composition of the present disclosure using any of a variety of known techniques. For example, water-soluble polymers can be “pre-plasticized” prior to incorporation into the composition. Alternatively, one or more of the components can be plasticized at the same time as they are blended together. Batch and/or continuous melt blending techniques can be employed to blend the components. For example, a mixer/kneader, Banbury mixer, Farrel continuous mixer, single-screw extruder, twin-screw extruder, roll mill, etc. can be used. One particularly suitable melt-blending device is a co-rotating, twin-screw extruder (e.g., USALAB twin-screw extruder available from Thermo Electron Corporation of Stone, England or an extruder available from Werner-Pfleiderer from Ramsey, N.J.). Such extruders can include feeding and venting ports and provide high intensity distributive and dispersive mixing. For example, the water-soluble polymer can be initially fed to a feeding port of the twin-screw extruder to form a composition. Thereafter, a plasticizer can be injected into the composition. Alternatively, the composition can be simultaneously fed to the feed throat of the extruder or separately at a different point along the length of the extruder. Melt blending can occur at any of a variety of temperatures, such as from about 30° C. to about 240° C., in some aspects, from about 40° C. to about 200° C., and in other aspects, from about 50° C. to about 180° C.

Plasticizers can be present in the water-dispersible, thermoplastic composition in an amount ranging from about 2 wt. % to about 50 wt. %, such as from about 3 wt. % to about 45 wt. %, and such as from about 5 wt. % to about 40 wt. %, based on the total weight of the composition. In some aspects, the plasticizer can be present in an amount of 10 wt. % or greater, such as from about 10 wt. % to about 35 wt. %, such as from about 10 wt. % to about 30 wt. %, and such as from about 10 wt. % to about 25 wt. % based on the total weight of the composition.

C. Fillers

Although the combination of the partially-hydrolyzed PVOH and plasticizer can achieve the desired water-solubility required for a water-dispersible, thermoplastic composition, it can still often be difficult to achieve a precise set of mechanical properties as desired for injected molded articles. In this regard, the composition can also contain one or more fillers. Due to its rigid nature, the amount of the filler can be readily adjusted to fine tune the composition to the desired degree of ductility (e.g., peak elongation) and stiffness (e.g., modulus of elasticity).

The filler of the present disclosure can include particles having any desired size, such as those having an average size of from about 0.5 to about 10 micrometers, in some aspects, from about 1 to about 8 micrometers, and in other aspects, from about 2 to about 6 micrometers. Suitable particles for use as a filler can include inorganic oxides, such as calcium carbonate, kaolin clay, silica, alumina, barium carbonate, sodium carbonate, titanium dioxide, zeolites, magnesium carbonate, calcium oxide, magnesium oxide, aluminum hydroxide, talc, etc.; sulfates, such as barium sulfate, magnesium sulfate, aluminum sulfate, etc.; cellulose-type powders (e.g., pulp powder, wood powder, etc.); carbon; cyclodextrins; and synthetic polymers (e.g., polystyrene).

In one particular aspect, the filler includes particles formed from calcium carbonate. If desired, calcium carbonate particles can be employed that have a purity of at least about 95 wt. %, in some aspects at least about 98 wt. %, and in other aspects at least about 99 wt. %. Such high purity calcium carbonate particles are generally fine, soft, and round, and thus provide a more controlled and narrow particle size for improving the properties of the composition. An example of such a high purity calcium carbonate is Caribbean micritic calcium carbonate, which is mined from soft and friable, finely-divided, chalk-like marine sedimentary deposits frequently occurring as surface deposits in the Caribbean region (e.g., Jamaica). Such calcium carbonates typically have an average particle size of about 10 micrometers or less, and desirably about 6 micrometers or less. Such calcium carbonates can be wet or dry ground, and classified into a narrow particle size distribution with round or spherical-shaped particles. One particularly suitable micritic calcium carbonate is available from Specialty Minerals under the designation MD1517.

Although not required, the filler can optionally be coated with a modifier (e.g., a fatty acid such as stearic acid or behenic acid) to facilitate the free flow of the particles in bulk and their ease of dispersion into the composition. The filler can be pre-compounded with such additives before mixing with the other components of the composition, or the additives can be compounded with the other components of the composition and fillers at the melt-blending step.

When present, the fillers can be present in an amount ranging from about 0.5 wt. % to about 35 wt. %, such as from about 1 wt. % to about 30 wt. %, such as from about 2 wt. % to about 25 wt. %, and such as from about 3 wt. % to about 20 wt. % based on the total weight of the water-dispersible, thermoplastic composition.

D. Coloring Agents

In addition, the water-dispersible, thermoplastic composition can contain one or more coloring agents (e.g., pigment or dye). Typically, a pigment refers to a colorant based on inorganic or organic particles that do not dissolve in water or solvents. Usually pigments form an emulsion or a suspension in water. On the other hand, a dye generally refers to a colorant that is soluble in water or solvents.

The pigment or dye can be present in an amount effective to be visible once the composition is formed into an injection molded article so that articles formed from the composition can have an aesthetically-pleasing appearance to the user. Suitable organic pigments include dairylide yellow AAOT (for example, Pigment Yellow 14 CI No. 21 095), dairylide yellow AAOA (for example, Pigment Yellow 12 CI No. 21090), Hansa Yellow, CI Pigment Yellow 74, Phthalocyanine Blue (for example, Pigment Blue 15), lithol red (for example, Pigment Red 52:1 CI No. 15860:1), toluidine red (for example, Pigment Red 22 CI No. 12315), dioxazine violet (for example, Pigment Violet 23 CI No, 51319), phthalocyanine green (for example, Pigment Green 7 CI No. 74260), phthalocyanine blue (for example, Pigment Blue 15 CI No. 74160), and naphthoic acid red (for example, Pigment Red 48:2 CI No. 15865:2). Inorganic pigments include titanium dioxide (for example, Pigment White 6 CI No. 77891), iron oxides (for example, red, yellow, and brown), chromium oxide (for example, green), and ferric ammonium ferrocyanide (for example, blue).

Suitable dyes that can be used include, for instance, acid dyes and sulfonated dyes including direct dyes. Other suitable dyes include azo dyes (e.g., Solvent Yellow 14, Dispersed Yellow 23, and Metanil Yellow), anthraquinone dyes (e.g., Solvent Red 111, Dispersed Violet 1, Solvent Blue 56, and Solvent Orange 3), xanthene dyes (e.g., Solvent Green 4, Acid Red 52, Basic Red 1, and Solvent Orange 63), azine dyes, and the like.

When present, the coloring agents can be present in the water-dispersible thermoplastic composition in an amount ranging from about 0.5 wt. % to about 20 wt. %, such as from about 1 wt. % to about 15 wt. %, such as from about 1.5 wt. % to about 12.5 wt %, and such as from about 2 wt. % to about 10 wt. % based on the total weight of the water-dispersible thermoplastic composition.

E. Other Optional Components

In addition to the components noted above, other additives can also be incorporated into the composition of the present disclosure, such as dispersion aids, melt stabilizers, processing stabilizers, heat stabilizers, light stabilizers, antioxidants, heat aging stabilizers, whitening agents, antiblocking agents, bonding agents, and lubricants. Dispersion aids, for instance, can also be employed to help create a uniform dispersion of the PVOH/plasticizer mixture and retard or prevent separation into constituent phases. Likewise, the dispersion aids can also improve the water dispersibility of the composition. Although any dispersion aid can generally be employed in the present disclosure, surfactants having a certain hydrophilic/lipophilic balance (“HLB”) can improve the long-term stability of the composition. The HLB index is well known in the art and is a scale that measures the balance between the hydrophilic and lipophilic solution tendencies of a compound. The HLB scale ranges from 1 to approximately 50, with the lower numbers representing highly lipophilic tendencies and the higher numbers representing highly hydrophilic tendencies. In some aspects of the present disclosure, the HLB value of the surfactants is from about 1 to about 20, from about 1 to about 15, or from about 2 to about 10. If desired, two or more surfactants can be employed that have HLB values either below or above the desired value, but together have an average HLB value within the desired range.

One particularly suitable class of surfactants for use in the present disclosure is that of nonionic surfactants, which typically have a hydrophobic base (e.g., a long chain alkyl group or an alkylated aryl group) and a hydrophilic chain (e.g., chain containing ethoxy and/or propoxy moieties). For instance, some suitable nonionic surfactants that can be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty (C.sub.8-C.sub.18) acids, condensation products of ethylene oxide with long chain amines or amides, condensation products of ethylene oxide with alcohols, fatty acid esters, monoglyceride or diglycerides of long chain alcohols, and mixtures thereof. In one particular aspect, the nonionic surfactant can be a fatty acid ester, such as a sucrose fatty acid ester, glycerol fatty acid ester, propylene glycol fatty acid ester, sorbitan fatty acid ester, pentaerythritol fatty acid ester, sorbitol fatty acid ester, and so forth. The fatty acid used to form such esters can be saturated or unsaturated, substituted or unsubstituted, and can contain from 6 to 22 carbon atoms, from 8 to 18 carbon atoms, or from 12 to 14 carbon atoms. In one particular aspect, mono- and di-glycerides of fatty acids can be employed in the present disclosure.

When employed, the dispersion aid(s) typically constitute from about 0.01 wt. % to about 15 wt. %, from about 0.1 wt. % to about 10 wt. %, from about 0.5 wt. % to about 5 wt. %, and from about 1 wt. % to about 3 wt. % based on the total weight of the water-dispersible thermoplastic composition.

Articles such as tampon applicators made from PVOH begin to disperse on contact with moisture or moist surfaces. In other applications, PVOH can used as an adhesive and will stick to surfaces such as the vaginal mucosal lining under moist conditions. This issue has limited the use of PVOH in flushable applications such as a tampon applicator.

There is still a need, however, to pursue a flushable tampon applicator that readily disperses in water in less than 60 minutes. To accomplish this, PVOH is the material of choice. An applicator made from PVOH alone, however, is not easily inserted under moist conditions. Eliminating this problem would allow for commercialization of a flushable tampon applicator.

Partially-hydrolyzed PVOH resins readily disperse on contact with water making them an excellent material for a flushable tampon applicator. During wet insertion, however, the outer surface of the applicator begins to disperse causing the PVOH to act as an adhesive, sticking to the mucosal lining.

Partially-hydrolyzed PVOH can be made less water dispersible by increasing the level of hydrolysis by reducing the remaining acetal groups to the alcohol form. This approach, however, eliminates the ability to be dispersible and therefore flushable.

It is desirable to hydrolyze only the surface of the PVOH article to a minimal depth, thereby maintaining the dispersibility of the whole article while at the same time reducing the dispersibility of the surface in contact with the moist surface.

One approach is to use electron beam radiation (e-beam) to cross-link the surface material of the PVOH article while maintaining the dispersibility of the entire article.

In one aspect, the resin used to produce the PVOH article is a blend of single low molecular weight partially-hydrolyzed PVOH and a plasticizer, such as glycerin, along with other optional additives.

In a particular example, the modified PVOH resin is a blend of 82 wt. % to 86 wt. % single low molecular weight partially-hydrolyzed PVOH, 11 wt. % to 13 wt. % glycerin, and 3 wt. % to 5 wt. % color and slip additives. The dispersal time in a modified slosh box test is less than 60 minutes.

The use of e-beam is desired to cross-link only to a minimal depth from the surface of the PVOH article. The necessary depth can be optimized by adjusting the e-beam dose and the distance of the surface from the e-beam, and by the use of cross-linking accelerants. Cross-linking accelerants include N,N′-methylene bisacrylamide (MBA). In some aspects, the depth of cross-linking obtained without the use of accelerants was 0.1 mm. A previous study showed that irradiated PVOH films without an activator were cross-linked at just 0.1%, whereas the presence of 4% MBA led to an 84% cross-linking at 50 kGy.

Exposing the surface of an article such as a tampon applicator to electron beam radiation can cross-link the surface material of the article and therefore improve wet insertion force as seen by changes in the physical properties of the material and an increase in contact angle of the surface of the material.

Reference now will be made in detail to various aspects of the disclosure, one or more examples of which are set forth below. Each example is provided by way of explanation of the disclosure, not limitation of the disclosure. In fact, it will be apparent to those skilled in the art that various modifications and variations can be made in the present disclosure without departing from the scope or spirit of the disclosure. For instance, features illustrated or described as part of one aspect, can be used on another aspect to yield a still further aspect. Thus, it is intended that the present disclosure covers such modifications and variations as come within the scope of the appended claims and their equivalents.

### V. EXAMPLES

A. Test Methods

Melt Flow Rate: The melt flow rate (“MFR”) is the weight of a polymer (in grams) forced through an extrusion rheometer orifice (0.0825-inch diameter) when subjected to a load of 2160 grams in 10 minutes, typically at 190° C. or 230°. Unless otherwise indicated, melt flow rate is measured in accordance with ASTM Test Method D1239 with a Tinius Olsen Extrusion Plastometer. It should be noted that the melt flow rate measured at 190° C. can be referred to as the melt flow index (MFI), while those measured at other temperatures are called melt flow rates (MFR).

Tensile Properties: Tensile properties were determined by following ASTM D638-10 guidelines. ASTM D638-10 Type V injection-molded test specimens were pulled via a MTS Mold 810 tensile frame with a 3,300 pound load cell. Five specimens were pulled from each example. The average values for peak stress (tensile strength), elongation at break, and modulus were reported. The maximum elongation that could be determined was 127% based on the tensile frame used, and the elongation was actually higher in the samples having 127% elongation readings.

Contact Angle: Contact angle was measured using Kruss Drop Shape Analyzer 100 which measured the contact angles of a water droplet (30 μL) 5 seconds after the water droplet landed on the surface of the disc sample. The average contact angle was determined on each disc surface at five different locations.

Flushability Assessment: Disintegration testing was performed as outlined in Guidance Document for Assessing the Flushability of Nonwoven Consumer Products (INDA and EDANA, 2006); Test FG 522.2 Tier 2—Slosh Box Disintegration Test. A round disc of each test resin is weighed and placed in 2 L of water maintained at 15° C. and agitated at 25-26 cycles per minute. The time for the material to disperse completely and pass through a 1 mm screen is recorded. After a maximum of 180 minutes, the test is stopped, any remaining pieces larger than 1 mm are collected, dried, and weighed. The percent weight remaining of the disc is recorded.

Coefficient of Friction: Coefficient of friction measurements were taken using a 32-07 Slip and Friction Tester supplied by Testing Machine Inc. Testing was done using a 100 g sled at a speed of 6 inches per minute with a static time of 1200 mS and a travel distance of 6 inches. Dry coefficient of friction was measured against steel. For wet coefficient of friction, 5 ml of distilled water was spread over the last 5 inches of travel before the test was started. The disc would start on dry steel and be pulled into the wet surface.

B. Materials

1. PVOH—Selvol 502—partially hydrolyzed 87%-89%; viscosity 3.0-3.7 cps—produced by Sekisui, Dallas, Tex.

### 2. Glycerin—Emery Cognis 916—Cognis Corporation, Cincinnati, Ohio

### 3. Colorant/Slip—SCC 85283—Standridge Color Corp., Social Circle, Ga.

4. N,N′-Methylene bisacrylamide—146072—Sigma-Aldrich, St. Louis, Mo.

Resin Compounding: In general, formulated resins were produced using the ZSK-30 co-rotating twin screw extruder with 7 heated sections and a resin compounding screw design. Resins were produced at a rate of 20 pounds per hour. PVOH and the color/slip agent was fed using a separate feeders into the main feed section. Glycerin was injected in section 3. The temperature profile per section, beginning at the main feed section was 90°, 130°, 160°, 190°, 190°, 180°, and 145° C. The melt pressure ranged between 30-50 psi with the extruder torque of between 35 to 45%. The extruded polymer was uniform in color and flowed well from the die. The strands were air cooled and pelletized.

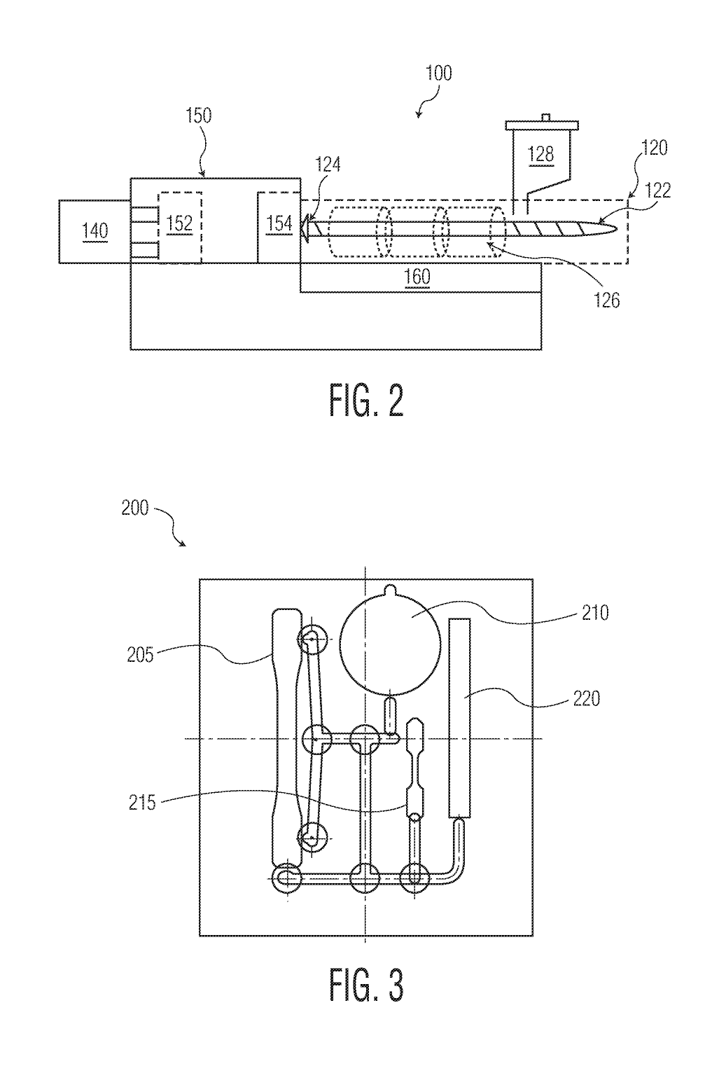

Injection Molding: The examples where processed on the Boy Machine 22D Injection Molder. This model has a 24.2 ton clamping force unit, a 24 mm plasticizing unit, and a shot size of 34 grams. FIG. 2 is a schematic of a basic injection molding machine 100. It shows the main components: the injection unit 120, the clamping unit 140, and the control panel 160. The injection molding cycle begins when the mold 150 closes, pairing the moveable platen 152 with the fixed platen 154. At this point, the screw 122 moves forward and injects the material through the nozzle 124 into the sprue, and the material fills the mold 150 (runners, gates, and cavities). During the packing phase, additional material is packed into the cavities. The material is cooled and solidifies in the mold while the screw 122 rotates counterclockwise backward, melting the plastic for the next shot using heating bands 126. New material is supplied by the hopper 128. The mold 150 opens and the parts are ejected. The next cycle begins when the mold 150 closes again.

The mold 200 used to produce specimens was an ASTM D638 standard test specimen mold from Master Precision Products, Inc., as illustrated in FIG. 3. This mold 200 contains a Tensile Type I specimen 205, a round disk 210, a Tensile Type V specimen 215, and an Izod bar 220.

E-Beam Irradiation: E-beam experiments were carried out at Comet Technologies, Shelton, Conn. The dose per sample was calculated using the equation:

K×I=D×S

### Where:

K=Constant, fixed value based on machine, air gap, and voltage

I=Current (mA)

D=Dose in kilograys (kGy)

S=Speed (m/min)

For this experiment the following were fixed:

Air gap between lamp and sample at 10 mm
Speed at 12 m/min

### Voltage at 200 kV

Penetration was 100% dose at depth of 0.1 mm

Disc samples received a single pass at the specified dose. To irradiate the three dimensional tubes, for each dose the tubes were passed under the emitter four times at half dose. On a single pass each side receives dosage based on reflection of electrons; therefore half a dose given four times simulates a turning tube.

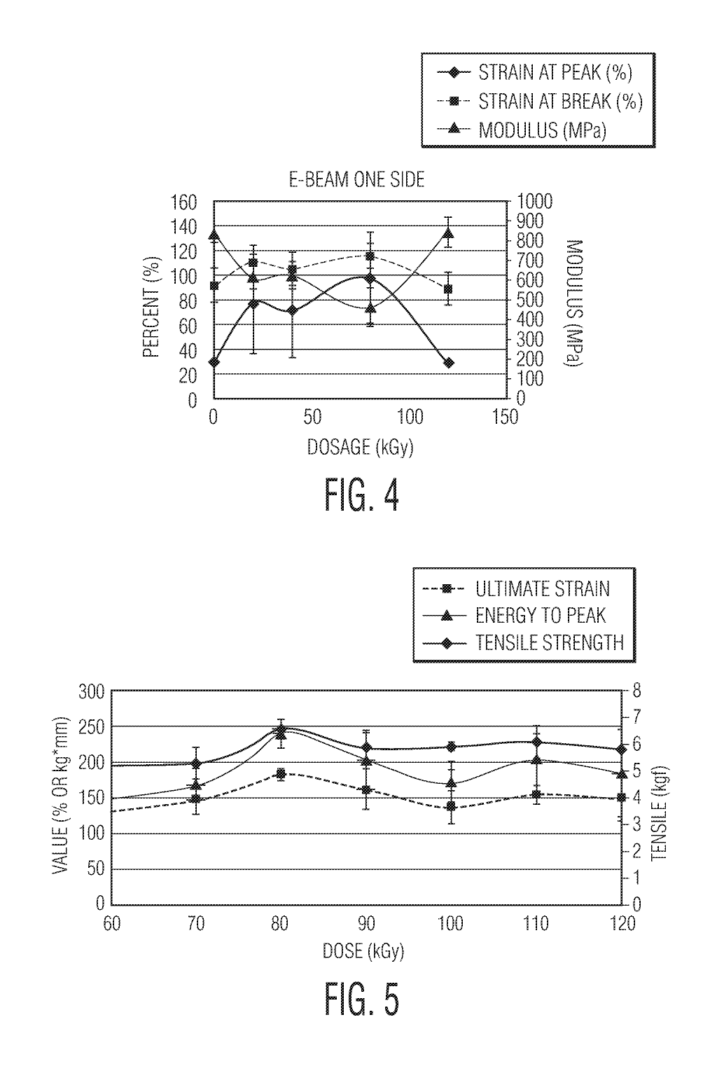

In the initial irradiation experiment, discs, tensile bar I, and Izod bars (see FIG. 3) were irradiated. One side of a disc was irradiated to test any changes in flushability and one side of a tensile bar was irradiated to measure the effect of physical properties. The initial doses tested were 20, 40, 80, and 120 kGy. An increase in peak strain at a dose of 80 kGy was seen, indicative of increased cross-linking, as illustrated in FIG. 4.

There was no change in the dissolution time as measured in the Slosh Box Disintegration Test. Some improvement was felt with wet insertion of sample bars receiving greater the 80 kGy. Numerical values were not recorded because the samples were not in the needed applicator tube shape.

The second e-beam experiment was performed to get more detail in the dosage curve. The peak in physical properties was seen at 80 kGy for testing of applicator tubes irradiated at various dosages (see FIG. 5), indicating that again there was some cross-linking. The results were similar to what was seen with exposed tensile bar samples.

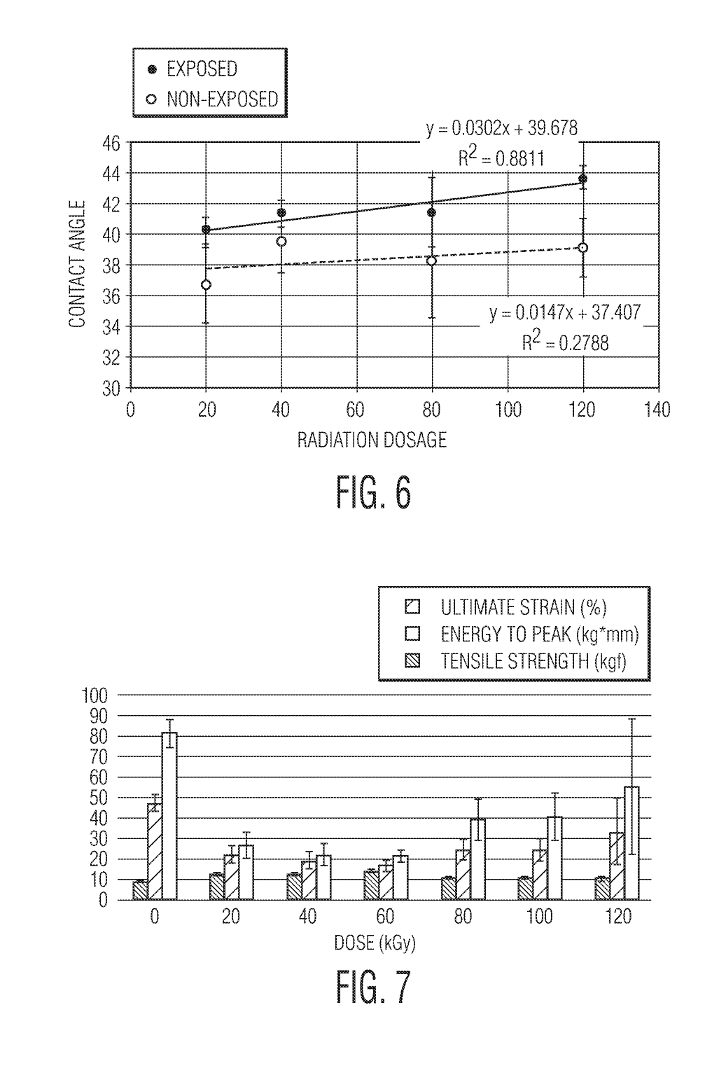

When comparing the contact angle of polymer discs where only one side was exposed to irradiation, there was a slight rise in contact angle indicating an increase in hydrophobicity of the surface exposed to e-beam radiation, as illustrated in FIG. 6.

To increase the level of cross-linking, the dose study was repeated with applicator tubes containing 4% methylene bisacrylamide as an accelerator. One side of the discs and four sides of the applicator tubes were exposed. In the case of the PVOH composition with an accelerator, the physical property effects were significantly different from applicators made from the PVOH composition without an accelerator, as illustrated in FIG. 7. The peak in tensile strength and the trough in elongation and energy all occurred at 60 kGy.

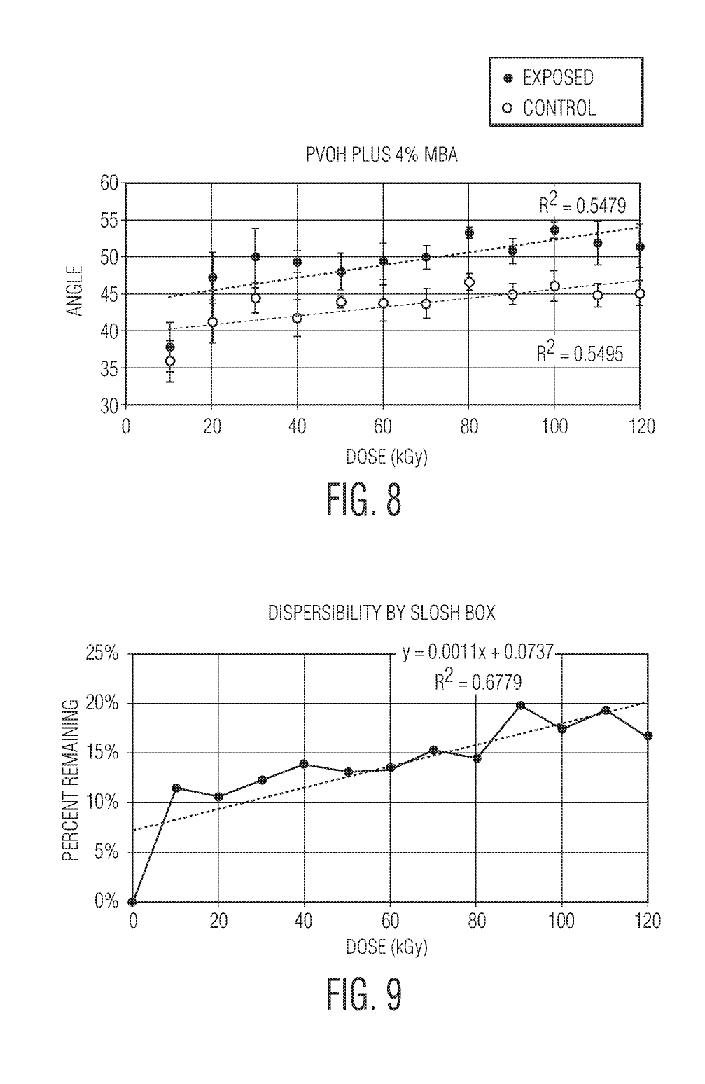

An increase in contact angle with dosage was found in the samples including an accelerator (see FIG. 8), and that increase was greater than that obtained with the PVOH composition exposed without an accelerator.

Testing of the dispersibility of discs exposed to various doses of radiation on one side showed a decrease in dispersibility with dose received. As the test proceeded, the non-exposed side dissolved first causing the disc to curl into a tube. After the maximum time of three hours, the remaining pieces were dried overnight and weighed. The percent material remaining increased with dosage, as illustrated in FIG. 9.

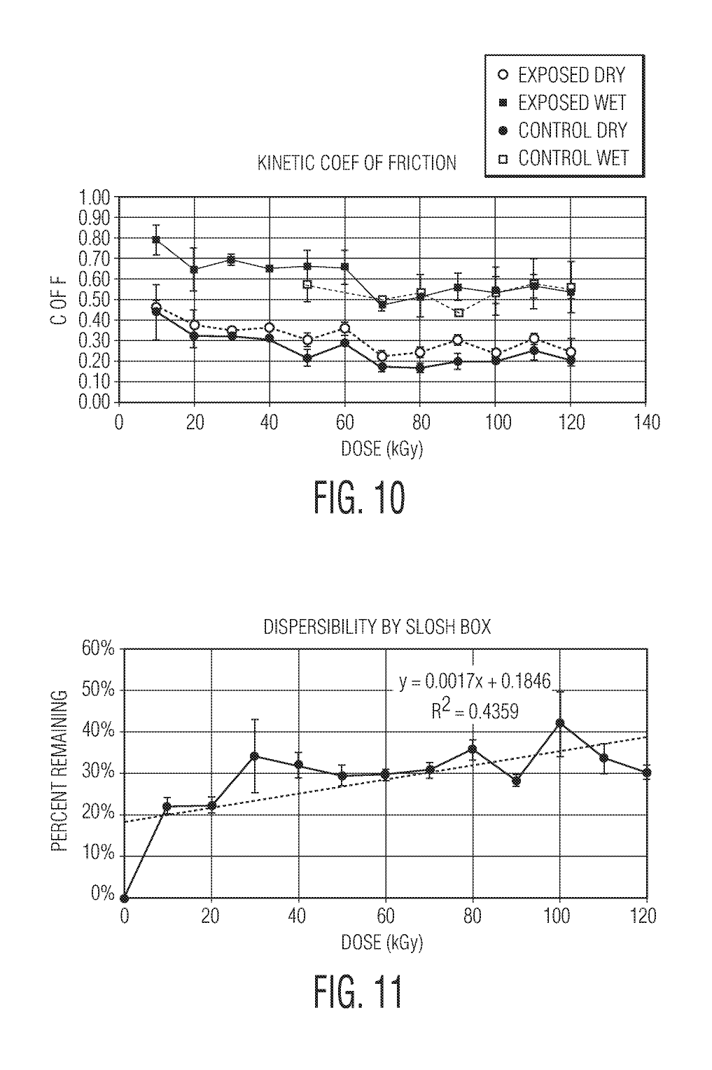

Finally, the dry and wet coefficient of friction of exposed and non-exposed discs were compared, as illustrated in FIG. 10. There does not appear to be a significant difference between exposed and non-exposed sides of the discs for dry or wet coefficient of friction. Following wet testing, however, the irradiated sides were less sticky than the non-exposed discs because they were slower to dissolve in water.

E-beam irradiation in the presence of an accelerator significantly affected dispersibility. To confirm this finding, the e-beam experiment was repeated by irradiating both sides of the PVOH composition discs including 4% MBA. The results confirmed the reduction in dispersibility of the material following irradiation, as illustrated in FIG. 11. The discs swelled for the first 90 minutes before breaking up into large pieces. Dispersibility was reduced from 100% in 30 minutes to 60% in 3 hours at an exposed dose of 100 kGy. This demonstrates the ability of e-beam treatment to alter PVOH solubility.

The inclusion of 4% MBA increased the cross-linking and acetyl hydrolysis of PVOH exposed to e-beam radiation. A slight increase in physical properties was seen at 60-80 kGy as expected from an increase in cross-links. The contact angle of the PVOH surface was slightly increased by exposure and the increase was greater with the addition of MBA. The exposed surface of PVOH with MBA became less dispersible as radiation dosage was increased, indicating an increase of acetyl hydrolysis that would cause the PVOH to absorb water but not disperse.

In a first particular aspect, an injection-molded article includes a water-dispersible injection-moldable composition including 82 wt. % to 86 wt. % partially-hydrolyzed polyvinyl alcohol (PVOH), 11 wt. % to 13 wt. % plasticizer, and 3 wt. % to 5 wt. % total colorant and slip additives, wherein the injection-molded article has an outer surface, and wherein the composition at the outer surface is surface cross-linked.

A second particular aspect includes the first particular aspect, wherein the composition at the outer surface has a higher degree of cross-linking than the rest of the composition in the injection-molded article.

A third particular aspect includes the first and/or second aspect, wherein the outer surface is surface cross-linked using electron beam radiation.

A fourth particular aspect includes one or more of aspects 1-3, wherein the composition further comprises a cross-linking accelerant.

A fifth particular aspect includes one or more of aspects 1-4, wherein the cross-linking accelerant is methylene bisacrylamide.

A sixth particular aspect includes one or more of aspects 1-5, wherein the composition at the outer surface has a lower water dispersibility than the rest of the composition in the injection-molded article.

A seventh particular aspect includes one or more of aspects 1-6, wherein the water dispersibility can be controlled by the amount and depth of surface cross-linking and by the overall surface coverage of the cross-linking.

An eighth particular aspect includes one or more of aspects 1-7, wherein the molded article is a tampon applicator.

A ninth particular aspect includes one or more of aspects 1-8, further including an outer tube for housing a tampon; and an inner tube, at least a portion of which extends into the outer tube, wherein the outer tube includes an outer, body-contacting surface, wherein the inner tube is moveable relative to the outer tube and configured to expel a tampon from the outer tube.

A tenth particular aspect includes one or more of aspects 1-9, wherein the resin blend is flushable according to Guidance Document for Assessing the Flushability of Nonwoven Consumer Products (INDA and EDANA, 2006); Test FG 522.2 Tier 2—Slosh Box Disintegration Test.

An eleventh particular aspect includes one or more of aspects 1-10, wherein the dispersal time in the modified slosh box disintegration test is less than 60 minutes.

In a twelfth particular aspect, a method for controlling the dispersibility of an injection-molded article having an outer surface includes formulating a water-dispersible injection-moldable composition including 82 wt. % to 86 wt. % partially-hydrolyzed polyvinyl alcohol (PVOH), 11 wt. % to 13 wt. % plasticizer, and 3 wt. % to 5 wt. % total colorant and slip additives; injection molding the single resin composition into the injection-molded article; and treating the outer surface to increase the cross-linking of the composition at the outer surface.

A thirteenth particular aspect includes the twelfth particular aspect, wherein the outer surface is treated using electron beam radiation.

In a fourteenth particular aspect includes the twelfth and/or thirteenth aspect, the composition further including a cross-linking accelerant.

A fifteenth particular aspect includes one or more of aspects 12-14, wherein the cross-linking accelerant is methylene bisacrylamide.

A sixteenth particular aspect includes one or more of aspects 12-15, wherein the composition at the outer surface has a lower water dispersibility than the rest of the composition in the injection-molded article.

A seventeenth particular aspect includes one or more of aspects 12-16, wherein the water dispersibility can be controlled by the amount and depth of surface cross-linking and by the overall surface coverage of the cross-linking.

An eighteenth particular aspect includes one or more of aspects 12-17, wherein the molded article is a tampon applicator.

A nineteenth particular aspect includes one or more of aspects 12-18, further including an outer tube for housing a tampon; and an inner tube, at least a portion of which extends into the outer tube, wherein the outer tube includes an outer, body-contacting surface, wherein the inner tube is moveable relative to the outer tube and configured to expel a tampon from the outer tube.

A twentieth particular aspect includes one or more of aspects 12-19, wherein the resin blend is flushable according to Guidance Document for Assessing the Flushability of Nonwoven Consumer Products (INDA and EDANA, 2006); Test FG 522.2 Tier 2—Slosh Box Disintegration Test, and wherein the dispersal time in the modified slosh box disintegration test is less than 60 minutes

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there can be additional elements other than the listed elements.

As various changes could be made in the above products without departing from the scope of the disclosure, it is intended that all matter contained in the above description and shown in the accompanying drawings shall be interpreted as illustrative and not in a limiting sense.

While the disclosure has been described in detail with respect to the specific aspects thereof, it will be appreciated that those skilled in the art, upon attaining an understanding of the foregoing, can readily conceive of alterations to, variations of, and equivalents to these aspects. Accordingly, the scope of the present disclosure should be assessed as that of the appended claims and any equivalents thereto.

1. An injection-molded article comprising:
a water-dispersible injection-moldable composition comprising:
82 wt. % to 86 wt. % partially-hydrolyzed polyvinyl alcohol (PVOH),
11 wt. % to 13 wt. % plasticizer, and
3 wt. % to 5 wt. % total colorant and slip additives,
wherein the injection-molded article has an outer surface, and wherein the composition at the outer surface is surface cross-linked.

2. The injection-molded article of claim 1, wherein the composition at the outer surface has a higher degree of cross-linking than the rest of the composition in the injection-molded article.

3. The injection-molded article of claim 1, wherein the outer surface is surface cross-linked using electron beam radiation.

4. The injection-molded article of claim 1, wherein the composition further comprises a cross-linking accelerant.

5. The injection-molded article of claim 4, wherein the cross-linking accelerant is methylene bisacrylamide.

6. The injection-molded article of claim 1, wherein the composition at the outer surface has a lower water dispersibility than the rest of the composition in the injection-molded article.

7. The injection-molded article of claim 6, wherein the water dispersibility can be controlled by the amount and depth of surface cross-linking and by the overall surface coverage of the cross-linking.

8. The injection-molded article of claim 1, wherein the molded article is a tampon applicator.

9. The injection-molded article of claim 8, further comprising an outer tube for housing a tampon; and
an inner tube, at least a portion of which extends into the outer tube, wherein the outer tube includes an outer, body-contacting surface, wherein the inner tube is moveable relative to the outer tube and configured to expel a tampon from the outer tube.

10. The injection-molded article of claim 1, wherein the resin blend is flushable according to Guidance Document for Assessing the Flushability of Nonwoven Consumer Products (INDA and EDANA, 2006); Test FG 522.2 Tier 2—Slosh Box Disintegration Test.

11. The injection-molded article of claim 10, wherein the dispersal time in the modified slosh box disintegration test is less than 60 minutes.

12. A method for controlling the dispersibility of an injection-molded article having an outer surface, the method comprising:
formulating a water-dispersible injection-moldable composition comprising:
82 wt. % to 86 wt. % partially-hydrolyzed polyvinyl alcohol (PVOH),
11 wt. % to 13 wt. % plasticizer, and
3 wt. % to 5 wt. % total colorant and slip additives;
injection molding the single resin composition into the injection-molded article; and
treating the outer surface to increase the cross-linking of the composition at the outer surface.

13. The method of claim 12, wherein the outer surface is treated using electron beam radiation.

14. The method of claim 12, the composition further comprising a cross-linking accelerant.

15. The method of claim 14, wherein the cross-linking accelerant is methylene bisacrylamide.

16. The method of claim 12, wherein the composition at the outer surface has a lower water dispersibility than the rest of the composition in the injection-molded article.

17. The method of claim 16, wherein the water dispersibility can be controlled by the amount and depth of surface cross-linking and by the overall surface coverage of the cross-linking.

18. The method of claim 12, wherein the molded article is a tampon applicator.

19. The method of claim 18, further comprising an outer tube for housing a tampon; and
an inner tube, at least a portion of which extends into the outer tube, wherein the outer tube includes an outer, body-contacting surface, wherein the inner tube is moveable relative to the outer tube and configured to expel a tampon from the outer tube.

20. The method of claim 12, wherein the resin blend is flushable according to Guidance Document for Assessing the Flushability of Nonwoven Consumer Products (INDA and EDANA, 2006); Test FG 522.2 Tier 2—Slosh Box Disintegration Test, and wherein the dispersal time in the modified slosh box disintegration test is less than 60 minutes.
